(19) United States
(12) Patent Application Publication
(10) Pub. No.: US 2011/0282127 A1
(43) Pub. Date: Nov. 17, 2011

(54) SYSTEM FOR RESTORING CARDIAC MUSCULAR ASYNCHRONIZED CONTRACTION MANNER IN A FAILING HEART

(76) Inventor: YINGJIE CUI, FLUSHING, NY (US)

(21) Appl. No.: 13/185,349

(22) Filed: Jul. 18, 2011

(51) Int. Cl. A61F 2/02

(52) U.S. Cl. 600/16

(57) ABSTRACT

An expandable elastic structure is introduced into the left ventricular chamber via intravascular catheter in a retrievable and safe manner, and having let anchors anchored to the layer of mid-myocardium of cardiac wall. The structure helps enhancing blood perfusion in the layer of both subendocardium and mid-myocardium and keeps the volume of both subendocardium and mid-myocardium in an expanded state, as such the expandable elastic structure helps restore cardiac muscular asynchronized contraction manner in a diseased heart of a patient. And eventually the expandable elastic structure prevents progressive remodeling process of a failing heart, and improves cardiac function.

### NOTICE OF MATERIAL SUBJECT TO COPYRIGHT PROTECTION

A portion of the disclosure of this patent document contains material which is subject to copyright protection under the copyright laws of the United States. The owner of the copyright rights has no objection to the facsimile reproduction by anyone of the patent document or the patent disclosure, as it appears in the United States Patent and Trademark Office patent file or records, but otherwise reserves all copyright rights whatsoever.

### CROSS-REFERENCE TO RELATED APPLICATIONS

Not Applicable

STATEMENT REGARDING FEDERALLY SPONSORED RESEARCH OR DEVELOPMENT

Not Applicable

THE NAMES OF THE PARTIES TO A JOINT RESEARCH AGREEMENT

Not applicable

REFERENCE OF MATERIAL SUBMITTED ON A COMPACT DISC

Not Applicable

### FIELD OF THE INVENTION

The present invention pertains to the field of treating heart failure and other cardiac disorder, and more particularly to methods and devices for preventing progressive remodeling process of a failing heart by restoring cardiac muscular asynchronized contraction manner.

### BACKGROUND OF THE INVENTION

Heart Failure (HF) is a major and growing public health problem in the United States. Approximately 5 million patients in this country have HF, and over 550 000 patients are diagnosed with HF for the first time each year. In 2001, nearly 53 000 patients died of HF as a primary cause. The number of HF deaths has increased steadily despite advances in treatment.

HF is a complex clinical syndrome that can result from any structural or functional cardiac disorder that impairs the ability of the ventricle to fill with or eject blood, and the majority of patients with HF show an impairment of left ventricle (LV) myocardial function. The impairment of LV myocardial function begins with some injury to, or stress on, the myocardium and is generally a progressive process, even in the absence of a new identifiable insult to the heart. The principal manifestation of such progression is a change in the geometry and structure of the LV, such that the chamber dilates and/or hypertrophies and becomes more spherical—a process referred to as cardiac remodeling. This change in chamber size and structure increases the hemodynamic stresses on the walls of the failing heart and depresses its mechanical performance. These effects, in turn, serve to sustain and exacerbate the remodeling process. Therefore, preventing the progressive remodeling process is seen as a major goal in the therapy of HF.

According to the authoritative Guidelines of American College of Cardiology Foundation/American Heart Association, although some therapies such as drug therapy show promising in some patients with HF, there is no known therapy in prior art therapy which can prevent progressive remodeling process of HF except the therapy of heart transplantation, because, at least, it was largely unknown the mechanism of the progressive remodeling process of HF. Furthermore, heart transplantation procedures are very risky, extremely invasive and expensive and are performed on a small percentage of patients due to multiple limitations. Therefore, substantial efforts have been made to find a novel treatment for HF.

Recent progress in the research of cardiac pumping mechanism provides an opportunity to develop a novel method to prevent the progressive remodeling process. Pathophysiology 17(2010)307 and The Thoracic & Cardiovascular Surgeon 58(2010)1, report a new mechanism of cardiac muscular contraction manner. It is that the three parts of heart wall including subendocardium, mid-myocardium, and subepicardium differ in contraction/relaxation manner, such that it appears as asynchronized contraction manner. For example, during systole, the powerful contraction of the mid-myocardium and subendocardium contributes to blood ejection; however, the subepicardium remains relaxed and is not committed to contraction simultaneously. During diastole, the subepicardium is committed to contraction while both subendocardium and mid-myocardium remains relaxed. This theory provides an important protection mechanism for heart to avoid enlargement in which subepicardium plays a key important role, and to avoid ischemia which is caused by abnormal myocardial contraction manner other than caused by coronary artery diseases.

On the other hand, alteration of cardiac muscular asynchronized contraction manner causes heart progressive remodeling process. First of all, it should be noticed that although subepicardium plays an important role in preventing excessive dilation of the heart during diastole, the subepicardium functions as such only when cardiac muscles contract in asynchronized manner. For example, subepicardium in simultaneous myocardial contraction manner does not counteract heart wall over expansion at the end of diastole and excessive dilation of the heart may occur in this case. Therefore, alteration of cardiac muscular asynchronized contraction manner may harm to this particular role of the subepicardium, and may cause ventricular dilation of the heart over time. Secondly, because cardiac muscular asynchronized contraction manner provides an important mechanism for blood perfusion in cardiac muscles, alteration of cardiac muscular asynchronized contraction manner is detrimental to blood perfusion in cardiac muscles and cause ischemia, the result of which, in turn, further deteriorate the capacity of cardiac muscular contraction for ejecting blood out of the heart. In summary, cardiac muscular asynchronized contraction manner plays an important role in maintaining heart function normally, and alteration of cardiac muscular asynchronized contraction manner causes progressive cardiac enlargement and cardiac pumping insufficiency.

As such, restoring cardiac muscular asynchronized contraction manner in diseased heart may bring benefits to prevent progressive remodeling process and even cure heart diseases of this cause at early intervention.

Therefore, there are substantial needs to develop methods and apparatus which may restore asynchronized contraction manner in a diseased heart of a patient, and which may be implantable in a retrievable and safe manner via intravascular catheter.

### SUMMARY OF THE INVENTION

It is therefore an object of the invention to provide methods and apparatus for treating HF and other heart disorder.

It is a further object of the invention to provide methods and apparatus for restoring cardiac muscular asynchronized contraction manner in a diseased heart of a patient.

It is also an object of the invention to provide methods and apparatus which are deliverable via an intravascular catheter in a retrievable and safe manner.

In accord with these objects, which will be discussed in detail below, the novel system of the present invention includes a cardiac support device, a delivery device and an intravascular catheter. The system delivers an implantable expandable cardiac support device into the left ventricular chamber with a delivery device via an intravascular catheter. Cardiac support device has anchors anchored to the layer of mid-myocardium of cardiac wall of left ventricle in the expanded state of the cardiac support device, and anchors are configured to allow blood flow via anchors and enhance blood perfusion in the cardiac muscles. Anchors keep the volume of both subendocardium and mid-myocardium in an expanded state, and eventually restore cardiac muscular asynchronized contraction manner and improve cardiac function in a diseased heart.

### BRIEF DESCRIPTION OF THE DRAWINGS

FIG. 1 is a schematic view of a cardiac support device in a collapsed state coupled to a delivery device inside an intravascular catheter.

FIG. 2 is a schematic view of a cardiac support device in an expanded state coupled to a delivery device inside an intravascular catheter.

FIG. 3A is a schematic view of a cardiac support device in a collapsed state.

FIG. 3B is a transverse cross sectional view of a cardiac support device in a collapsed state.

FIG. 4 is a schematic view of a cardiac support device in an expanded state

FIG. 5A is a schematic front view of an anchor.

FIG. 5B is a transverse cross sectional view of a proximal binding site of an anchor of FIG. 5A.

FIG. 5C is a transverse cross sectional view of an insertable part of an anchor of FIG. 5A.

FIG. 6A is a schematic rear view of an anchor.

FIG. 6B is a transverse cross sectional view of a proximal binding site of an anchor of FIG. 6A.

FIG. 6C is a transverse cross sectional view of an insertable part of an anchor of FIG. 6A.

FIG. 7A is a schematic upper-side view of a left part of an anchor.

FIG. 7B is a transverse cross sectional view of a proximal binding site of an anchor of FIG. 7A.

FIG. 7C is a transverse cross sectional view of an insertable part of an anchor of FIG. 7A.

FIG. 8A is a schematic upper-side view of a right part of an anchor.

FIG. 8B is a transverse cross sectional view of a proximal binding site of an anchor of FIG. 8A.

FIG. 8C is a transverse cross sectional view of an insertable part of an anchor of FIG. 8A.

FIG. 9 is a schematic view of a cardiac support device being inserted into a left ventricle by a delivery device via an intravascular catheter.

FIG. 10 is a longitudinal cross sectional view of a cardiac support device anchored to a ventricular wall via anchors in an expanded state.

FIG. 11 is a schematic view of a delivery device.

### DETAILED DESCRIPTION OF THE INVENTION

As mentioned above, the present invention includes devices and methods for restoring cardiac muscular asynchronized contraction manner in a diseased heart for treating dysfunctional left ventricle.

Before the present invention is described in detail, it is to be understood that this invention is not limited to particular embodiments and applications described. It is also to be understood that the terminology used herein is for the purpose of describing particular embodiments only, and is not intended to be limiting, since the scope of the present invention will be limited only by the appended claims. Furthermore, the methods recited herein may be carried out in any order of the recited events which is logically possible, as well as in the recited order of events. Although any methods and materials similar or equivalent to those described herein can also be used in the practice or testing of the present invention, the preferred methods and materials are now described.

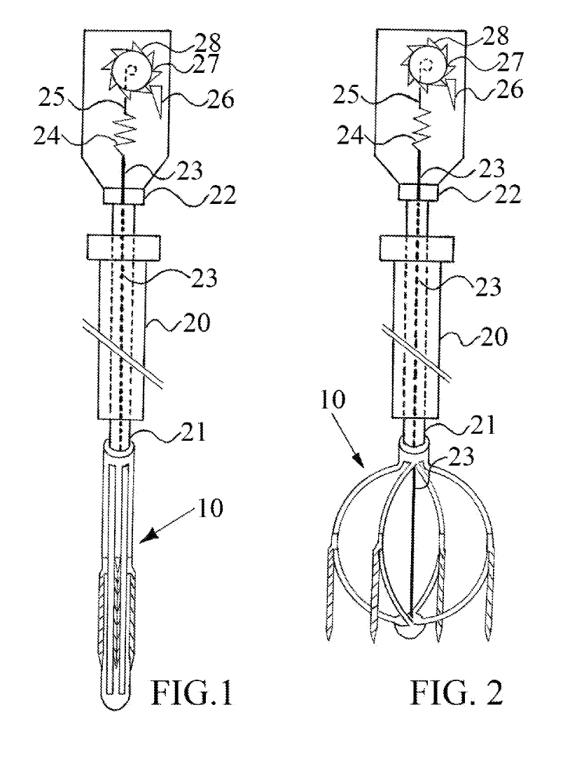

FIG. 1 and FIG. 2 illustrate a system for restoring cardiac asynchronized contraction manner which is used for the treatment of heart diseases which include but not limited to heart failure. The system includes a cardiac support device 10 and a delivery device 30 and an intravascular catheter 20.

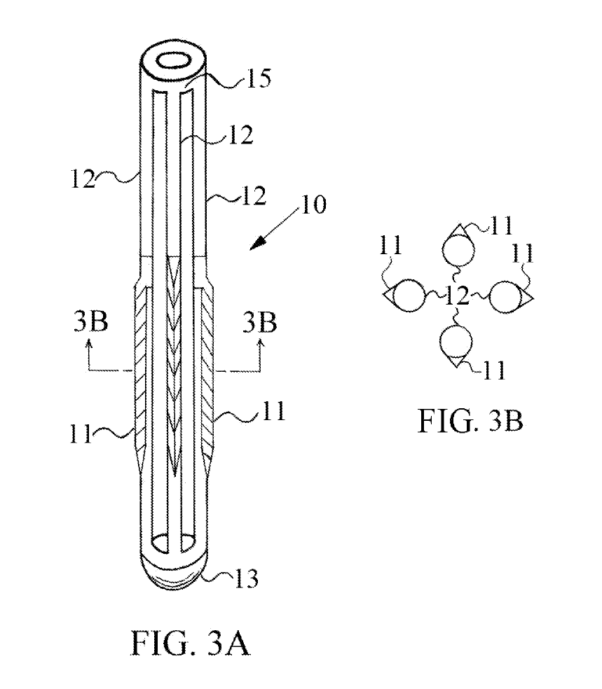

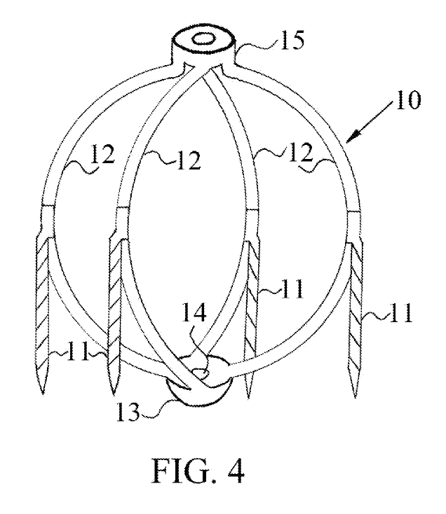

As shown in FIG. 3A and FIG. 3B and FIG. 4, said cardiac support device 10 comprises a plurality of elastic arms 12. Said elastic arms 12 are made of resilient materials including but not limited to Nitinol Alloys. The number of said elastic arms 12 can vary from three to more than ten, which depends on the nature of heart diseases and characteristics of resilient materials. Also, the length and width of said elastic arms 12 may vary based on the size of a ventricular chamber 100 and characteristics of resilient materials. In addition, the shape of said elastic arms 12 is not limited to cylindrical as shown in the embodiment of the present invention. The proximal end of said elastic arms 12 is joined together by a proximal circumferential band 15 extending therebetween, and said proximal circumferential band 15 is made of metal including but not limited to Titanium. The distal end of said elastic arms 12 is joined by a distal cover 13 which is made of metal including but not limited to Titanium , and there is a hole 14 in the center of said distal cover 13. There are threads inside said hole 14, in which a shaft 23 of a delivery device 30 is capable of connecting and locking to said distal cover 13 releasably through threads of said hole 14, as such upon completion of inserting and installing said cardiac support device 10 inside a ventricular chamber 100, said shaft 23 of said delivery device 30 is capable of being disconnected to said distal cover 13 and being removed from said ventricular chamber 100.

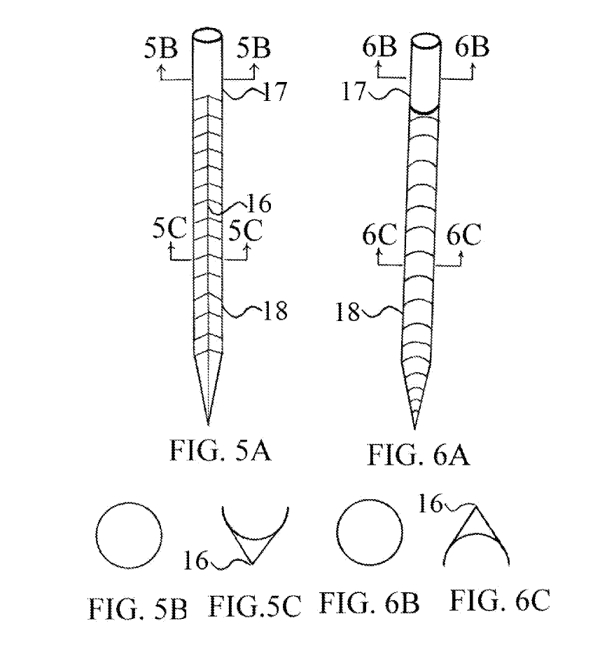

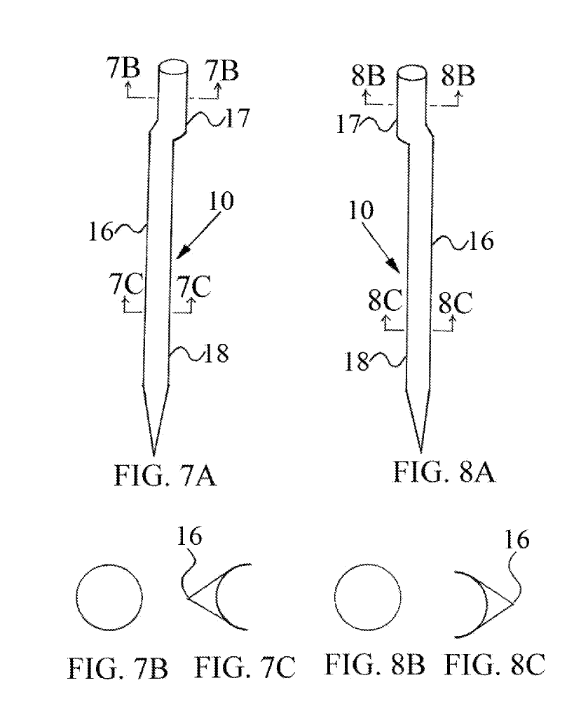

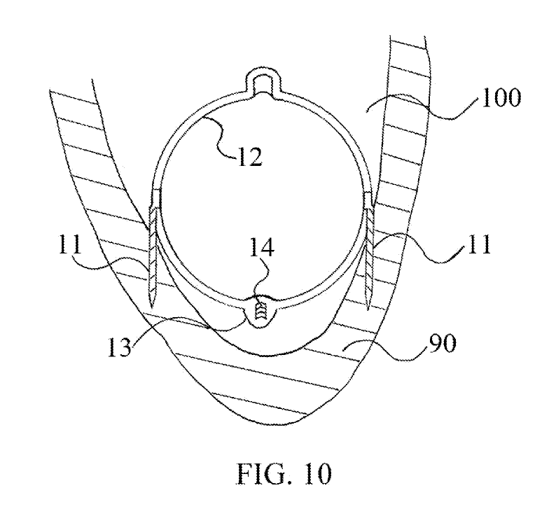

As shown in FIG. 4, FIG. 5A-C, FIG. 6A-C, FIG. 7A-C, FIG. 8A-C, each of said elastic arms 12 has an anchor 11 attached longitudinally. Said anchor 11 is made of non-resilient metal including but not limited to Titanium. Said anchor 11 comprises a proximal binding site 17 where said anchor 11 is attached to said elastic arms 12, and a distal insertable part 18 which is inserted into the layer of mid-myocardium 90 of cardiac muscles as shown in FIG. 10. Said proximal binding site 17 of said anchor 11 is bound to the middle part of said elastic arms 12. Each of said proximal binding sites 17 of said anchor 11 is permanently attached to each of said elastic arms 12 as shown in the embodiment of this patent document. Said proximal binding site 17 of said anchor 11 produces inward traction toward the center of ventricular chamber 100 in the expanded state of said cardiac support device 10. This inward traction of said binding site 17 of said anchor 11 is produced by mechanical energy of said elastic arms 12 in an expanded state of said cardiac support device 10, and transferred to both subendocardium and mid-myocardium via said insertable part 18 of said anchor 11 so that the volume of both subendocardium and mid-myocardium is kept in an expanded state during systole and diastole, in particular, during diastole. The extent of the expanded state of both subendocardium and mid-myocardium is predetermined by the length, diameter, elasticity, and number of said elastic arms 12.

Further, the midline of the projection surface of said distal insertable part 18 of said anchor 11 has a sharp edge 16. Said sharp edge 16 is capable of having said anchor 11 inserted into the layer of mid-myocardium 90 of cardiac muscles, and also said sharp edge 16 prevents said insertable part 18 of said anchor 11 permanently binding to cardiac muscles so as to keep blood flow channel patency through said anchor 11. Consequently, the expanded state of both subendocardium and mid-myocardium helps blood perfusion in the layer of subendocardium and mid-myocardium, and restores cardiac muscular asynchronized contraction manner in a diseased heart.

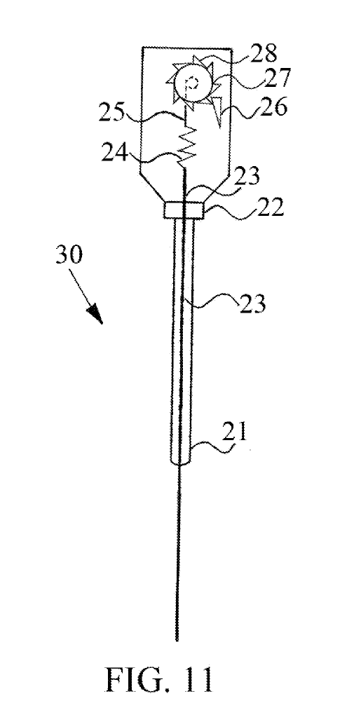

Said cardiac support device 10 is delivered into said ventricular chamber 100 by said delivery device 30. Said cardiac support device 10 is releasably coupled to said delivery device 30 through interlocking between said distal cover 13 and the distal end portion of said shaft 23. As shown in FIG. 11, said delivery device 30 comprises said shaft 23 which is positioned into the center line of said cardiac support device 10 and having a distal end portion releasably coupled to said distal cover 13 of said cardiac support device 10 as described above, wherein said shaft 23 is made of metal including but not limited to Titanium, and wherein the proximal end portion of said shaft 23 is connected to the distal end of a resilient structure 24; a shaft releaser 22 having let said shaft 23 pass through the center line of said shaft releaser 22 and integrate together so that said shaft 23 can be screwed off and disconnected from said distal cover 13 of said cardiac support device 10 by rotating said shaft releaser 22, wherein said shaft releaser 22 is made of metal or plastic; a shaft cover 21 extending from said shaft releaser 22 to said proximal circumferential band 15, wherein said shaft cover 21 is made of metal including but not limited to Titanium, and wherein said shaft cover 21 is used for counteracting the traction of said shaft 23 for the purpose of expansion of said cardiac support device 10; a resilient structure 24 comprising metal coil springs which provide protection to heart tissue and said cardiac support device 10 when excessive traction is imposed on said shaft 23; a connecting element 25 which is made of nylon fiber and provides connection between the proximal end of said resilient structure 24 and a reel 27; said reel 27 which is made of metal or plastic and is capable of winding up said connecting element 25 on reel in order to expand said cardiac support device 10; a break 26 which is made of metal or plastic and is capable of keeping said elastic arms 12 expandable or collapsible in a desired extent by interacting with the teeth 28 of said reel 27.

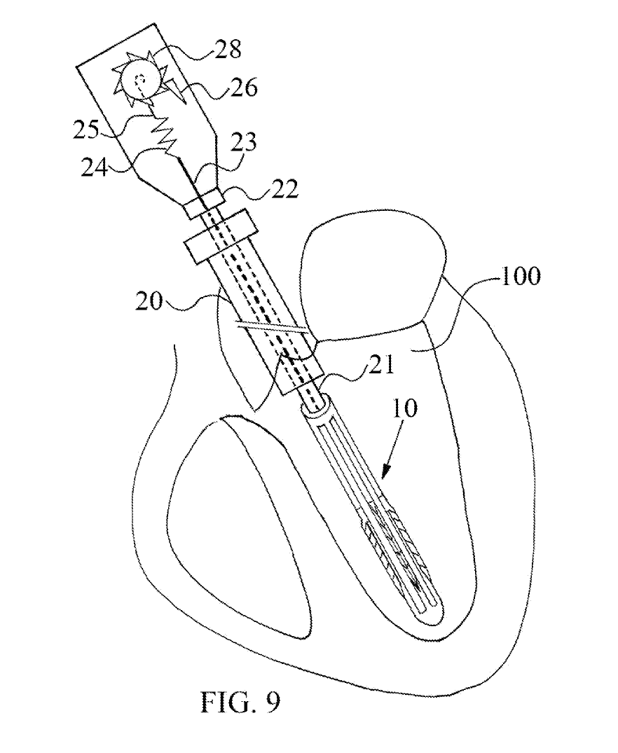

An intravascular catheter 20 is made of polymer, and the size of said intravascular catheter 20 is in the same range as other percutaneous cardiac procedures, using sizes in the range of 18 Fr to 28 Fr. Said intravascular catheter 20 is deployed into left ventricular chamber 100 percutaneously through right carotid artery and aortic valve using a common guide wire (not shown), followed by the insertion of said cardiac support device 10 coupled to said delivery device 30 as shown in FIG. 9. In addition, said cardiac support device 10 can be retrieved from the anchored position by simply pulling back said cardiac support device 10 by a wire (not shown) via said intravascular catheter 20.

What is claimed is:

1. A system capable of restoring cardiac muscular asynchronized contraction manner in a failing heart, comprising:
an intravascular catheter that is percutaneously insertable into a ventricular chamber of a heart;
a cardiac support device deliverable via said intravascular catheter into a ventricular chamber in a collapsed state and anchorable via an anchor to a wall of the ventricular chamber in an expanded state, comprising a plurality of longitudinal elastic arms physically coupled together a proximal and a distal end of said elastic arms, the proximal end of said elastic arms being joined by a proximal circumferential band extending therebetween and the distal end of said elastic arms being joined by a distal cover, said anchor having a configuration adapted to allow blood flow via said anchor in an anchored state; and
a delivery device releasably coupled to said cardiac support device, comprising:
a shaft positioning into the center line of said cardiac support device and having a distal end portion releasably coupled to said distal cover of said cardiac support device;
a shaft releaser having let said shaft pass through the center line of said shaft releaser and disconnecting said shaft from said distal cover of said cardiac support device by rotating said shaft releaser;
a resilient structure having distal end portion connected to the proximal end of said shaft, comprising coil springs; and
a reel winding or unwinding a non-resilient connecting element which is connecting said reel to said resilient structure.

2. The system of claim 1, wherein said elastic arms are constructed of resilient material.

3. The system of claim 1, wherein said anchor is constructed of non-resilient material.

4. The system of claim 1, wherein said anchor is configured to be insertable into the layer of mid-myocardium of the ventricular wall.

5. The system of claim 1, wherein said distal cover has a hole and has stationary threads inside the hole.

6. The system of claim 1, wherein the distal end portion of said shaft has threads around outside.

7. The system of claim 1, wherein said shaft of said delivery device is coupled to said distal cover of said cardiac support device by screwing said shaft into said distal cover.

8. The system of claim 1, wherein said delivery device is capable of disconnecting from said cardiac support device by releasing said shaft from said distal cover.

9. A system capable of restoring cardiac muscular asynchronized contraction manner in a failing heart, comprising:
an intravascular catheter that is percutaneously insertable into a ventricular chamber of a heart;
a cardiac support device deliverable via said intravascular catheter into a ventricular chamber in a collapsed state and anchorable via an anchor to a wall of the ventricular chamber in an expanded state, comprising a plurality of longitudinal elastic arms physically coupled together a proximal and a distal end of said elastic arms, the proximal end of said elastic arms being joined by a proximal circumferential band extending therebetween and the distal end of said elastic arms being joined by a distal cover, each of said elastic arms having at least one anchor in the middle part of each of said elastic arms, said anchor having a configuration adapted to be anchorable into the layer of mid-myocardium of the ventricular wall; and
a delivery device releasably coupled to said cardiac support device, comprising:
a shaft positioning into the center line of said cardiac support device and having a distal end portion releasably coupled to said distal cover of said cardiac support device;
a shaft releaser having let said shaft pass through the center line of said shaft releaser and disconnecting said shaft from said distal cover of said cardiac support device by rotating said shaft releaser;
a resilient structure having a distal end portion connected to the proximal end of said shaft, comprising coil springs, wherein said resilient structure is adapted to counteract excessive forces imposed on said cardiac support device in the expanded state of said cardiac support device; and
a reel winding or unwinding a non-resilient connecting element which is connecting said reel to said resilient structure.

10. The system of claim 9, wherein said cardiac support device has pillar shape in a collapsed state and has spherical or ellipsoid shape in an expanded state.

11. The system of claim 10, wherein said cardiac support device is anchorable into mid-myocardium via longitudinal said anchor in an expanded state.

12. The system of claim 11, wherein the proximal end portion of each of said anchor is firmly attached to each of said elastic arms and the distal end portion of each of said anchor is detachable from each of said elastic arms in the expanded state of said cardiac support device.

13. The system of claim 12, wherein said anchor is insertable into mid-myocardium of the ventricular wall in the state of said anchor attaching to the ventricular wall in the expanded state of said cardiac support device.

14. The system of claim 13, wherein said shaft of said delivery device is released from said distal cover of said cardiac support device when installation of said cardiac support device inside a ventricular chamber is completed.

15. A system capable of restoring cardiac muscular asynchronized contraction manner in a failing heart, comprising:
an intravascular catheter that is percutaneously insertable into a ventricular chamber of a heart;
a cardiac support device deliverable via said intravascular catheter into a ventricular chamber in a collapsed state and anchorable longitudinally via an anchor into the layer of mid-myocardium of the ventricular wall in an expanded state in a retrievable manner, comprising a plurality of longitudinal elastic arms physically coupled together a proximal and a distal end of said elastic arms, each of said elastic arms having at least one anchor in the middle part of each of said elastic arms, said anchor anchorable longitudinally into the layer of mid-myocardium of the ventricular wall, said anchor having a configuration adapted to allow blood flow via anchor, said anchor having inward traction toward center of the ventricular chamber produced by the expansion of said elastic arms; and
a delivery device releasably coupled to said cardiac support device, comprising:
a shaft positioning into the center line of said cardiac support device and having a distal end portion releasably coupled to said distal cover of said cardiac support device;
a shaft releaser having let said shaft pass through the center line of said shaft releaser and disconnecting said shaft from said distal cover of said cardiac support device by rotating said shaft releaser;
a resilient structure having distal end portion connected to the proximal end of said shaft, comprising coil springs, wherein said resilient structure is adapted to counteract excessive forces imposed on said cardiac support device in the expanded state of said cardiac support device; and
a reel winding or unwinding a non-resilient connecting element which is connecting said reel to the proximal end of said resilient structure.

16. The system of claim 15, wherein said elastic arms are expandable from a collapsed state inside a ventricular chamber by winding up said non-resilient connecting element onto said reel and collapsible from an expanded state by unwinding said non-resilient connecting element from said reel.

17. The system of claim 16, wherein said elastic arms are configured to absorb mechanical energy during diastole and emit mechanical energy during systole.

18. The system of claim 17, wherein the mechanical energy of said elastic arms are transferred to both subendocardium and mid-myocardium via longitudinal said anchor.

19. The system of claim 18, wherein the muscle volume of both subendocardium and mid-myocardium is kept in an expanded state during systole and diastole, in particular, during diastole, wherein the extent of the expanded state of the muscle volume of both subendocardium and mid-myocardium is predetermined so as to enhance blood perfusion in both subendocardium and mid-myocardium and restore cardiac muscular asynchronized contraction manner in myocardium via longitudinal said anchor.

20. The system of claim 15, wherein said cardiac support device is retrievable via said intravascular catheter.
